(19) United States
(12) Patent Application Publication
(10) Pub. No.: US 2021/0315626 A1
(43) Pub. Date: Oct. 14, 2021

(54) CRYOABLATION APPARATUS AND METHOD

(71) Applicant: PIEDMONT MEDSYSTEMS (ZHUHAI) CO., LTD., Zhuhai, Guangdong (CN)

(72) Inventors: Jiahua Xiao, Zhuhai, Guangdong (CN); Dongbo Su, Zhuhai, Guangdong (CN); Alan De La Rama, Zhuhai, Guangdong (CN); Cary Kunihiko Hata, Zhuhai, Guangdong (CN)

(73) Assignee: PIEDMONT MEDSYSTEMS (ZHUHAI) CO., LTD., Zhuhai, Guangdong (CN)

(21) Appl. No.: 17/250,446

(22) PCT Filed: Aug. 1, 2018
(86) PCT No.: PCT/CN2018/097905

§ 371 Date: Jan. 22, 2021

(30) Foreign Application Priority Data
Jul. 23, 2018 (CN) 201810812250.3

(51) Int. Cl. A61B 18/02

(52) U.S. Cl. CPC A61B 18/02; A61B 2018/0022; A61B 2018/0262; A61B 2018/0212

(57) ABSTRACT

A cryoablation device comprises a freezing balloon, a catheter, a storage tank, a delivery pipeline, a recovery pipeline, and a freezing element. Within the freezing balloon circulates a freezing substance, such as a low pressure liquid or gas or a gas-liquid mixture. A cryoablation method comprises the following four steps: pre-freezing, ablation, recovery, and rewarming. Advantages of the cryoablation device and method provided herein: low risk factor, easy to operate, convenient to use, exhibits high freezing efficiency, and temperature control accuracy.

This application is the National Stage of, and therefore claims the benefit of and priority to, International Application No. PCT/CN2018/097905, filed on Aug. 1, 2018, entitled “Cryoablation Apparatus and Cryoablation Method,” which was published as International Publication No. WO 2020/019362 A1 on Jan. 30, 2020, and has a priority date of Jul 23, 2018, based on Chinese Patent Application No. CN 201810812250. Both of the above applications are commonly assigned with this National Stage application, and the entireties thereof are hereby incorporated herein by reference.

### TECHNICAL FIELD

The present invention relates to the technical field of medical equipment, in particular to a cryoablation device and a cryoablation method.

### BACKGROUND TECHNIQUE

Cryoablation is a surgical medical technique that uses freezing to eliminate target tissues, and is mostly used to treat tumors, atrial fibrillation and other diseases. The principle is to use low-temperature equipment to make the target tissue undergo a process of cold, freezing, and rewarming, thereby causing irreversible damage or necrosis to the cells. The cryoablation equipment generally includes a cryoablation generator (host) and a cryo-balloon part. The cryo-ablation generator (host) is responsible for providing cold-carrying medium for the cryo-balloon. When in use, the cryo-balloon is installed at the tip of the catheter and extended into the body. The cryoablation generator (host) passes the cold-carrying medium from the catheter into the cryo-balloon to cool it down, and then cryoablate the target tissue.

The cryoablation generator in the prior art generally adopts high-pressure gas throttling refrigeration method. When the high-pressure gas flows through the small hole and then adiabatic throttling, the temperature tends to change the pressure drops, and the gas adiabatic throttling is used The effect can complete the cooling treatment process of cryoablation. However, the high-pressure gas throttling method has certain defects in actual use: First, the high-pressure gas has a high risk factor. Since the cryoablation terminal balloon is generally in the human body, once the gas pressure is too high, the balloon may rupture, posing a great safety threat to the patient; secondly, high-pressure gas is a consumable, which needs to be supplemented and is inconvenient to use; again, the use of equipment The requirements for the operator are high, and the operation needs to be accompanied by a professional technician during the operation.

### SUMMARY OF THE INVENTION

Therefore, the technical problem to be solved by the present invention is to overcome the defects of high risk factor, fast working fluid consumption, and inconvenience in use of the cryoablation device in the prior art, and to provide a low risk factor, extremely low working fluid loss, Easy to use cryoablation device.

Another technical problem to be solved by the present invention is to overcome the defects of the prior art cryoablation method of high risk factor and difficulty in operation, and further provide a cryoablation method with low risk factor and simple operation.

In order to solve the above technical problems, the technical solutions provided by the present invention are as follows:

A cryoablation device includes:

The cryo-balloon, in which a cold medium is circulated, is suitable for contact with human tissue and cryoablation;

The catheter, which is watered to the freezing balloon, has a medium input end and a medium output end, and is suitable for inputting and outputting cold medium into the freezing balloon;

Medium storage tank, in which cold medium is stored;

The medium recovery pipeline, one end is connected with the medium storage tank, and the other end is connected with the medium output end of the pipe;

The refrigeration component is connected in series with the medium supply pipeline, and is suitable for refrigeration and heat exchange of the refrigerating medium in the medium supply pipeline.

As a preferred technical solution, the refrigeration assembly includes:

Cooling capacity generating device, used to generate cooling capacity:

The first refrigeration exchange device is installed on the medium supply pipeline, the refrigeration generating device provides refrigeration to the first refrigeration exchange device, and the first refrigeration exchange device is used for The cooling medium in the quantity exchange device performs refrigeration and heat exchange;

As a preferred technical solution, the refrigeration assembly further includes:

The second cold quantity exchange device has a hot fluid passage installed on the medium supply pipe and a cold fluid passage installed on the medium recovery pipe, and cold energy is generated between the cold fluid passage and the hot fluid passage Exchange, pre-cooling the cooling medium flowing through the hot fluid channel;

The hot fluid passage is concerted between the medium storage tank and the first cold quantity exchange device.

As a preferred technical solution, it also includes:

A bypass pipe which is connected to the medium supply pipeline and the medium recovery pipeline, and enables the medium supply pipeline and the medium recovery pipeline to form a pre-cooling circuit of the serial medium storage tank and the first cold capacity exchange device;

And the bypass pipe and the medium supply pipeline communicate with each other through a first three-way valve.

As a preferred technical solution, the refrigeration assembly further includes:

The cold storage device is installed on the medium recovery pipeline, communicates with the first cold energy exchange device by a bypass pipe and is writable for storing the cold energy flowing out of the first cold energy exchange device.

As a preferred technical solution, the refrigeration assembly further includes:

The heat insulation device has a heat insulation cavity suitable for reducing or isolating heat conduction from the outside, and the cold output ends of the first cold energy exchange device, the second cold energy exchange device, the cold storage device and the cold energy generation device are located in the insolation Hot cavity.

As a preferred technical solution, the heat insulation device is a box body, and a vacuum pumping device communicating with the heat insulation cavity is installed on the heat insulation device.

As a preferred technical solution, the heat insulation device is a box, and the heat insulation cavity is filled with a heat insulation material

As a preferred technical solution, it further includes a reheating circuit for transporting the cold-carrying medium in the medium storage tank to the medium input end of the catheter in the cryoablation device.

As a preferred technical solution, the reheating circuit includes:

Rehearing tube

The medium inlet end of the reheating pipe is concerted with the side installed on the medium supply pipeline that dons not enter the first cold quantity exchange device by a second three-way valve.

As a preferred technical solution, it further include a reheating circuit for heating the cold-carrying medium in the medium storage tank and then delivering it to the medium input end of the catheter in the cryoablation device.

As a preferred technical solution, the reheating circuit includes:

Reheating tube with a heating device connected in series;

The medium inlet end of the reheating pipe is connected with the upstream of the medium inlet port that enters the hot fluid channel by a second three-way valve.

As a preferred technical solution, the reheating circuit further includes:

The rewarming return line is used to connect the medium output end of the catheter in the cryoablation device with the medium storage tank.

As a preferred technical solution, the reheating return pipeline includes:

The reheating return pipe is connected with the medium recovery pipeline at both ends, and is connected in parallel with the second cold capacity exchange device;

The medium inlet end of the reheating return pipe is connected with the medium supply pipeline by a third three-way valve.

As a preferred technical solution, the refrigeration assembly further includes:

The heat insulation device has a heat insulation cavity suitable for reducing or isolating heat conduction from the outside, and the cold output ends of the first cold energy exchange device, the second cold energy exchange device, the cold storage device and the cold energy generation device are located Hot cavity

The rehearing return pipe is outside the heat insulation device.

As a preferred technical solution a pumping device is concerted in series to the medium supply pipeline or the medium recovery pipeline, and the pumping device is suitable for providing power for the flow of the cooling medium.

A cryoablation method includes the following steps:

Pre-cooling, passing the cooling medium cooling cycle into the cooling capacity generator for cooling

Ablation the pre-cooled cold-carrying medium is recirculated into the cold generation device for cooling again, and then passed into the target tissue of the human body, so that the cold-carrying medium and the target tissue can exchange cold, so that the target Perform cryoablation of the targeted tissue after cooling down the tissue;

After rewarming, stop passing the cooled cold carrier medium into the human body to raise the temperature of the target tissue.

In a preferred technical solution, in the recovery step, the cold-carrying Medium after cold exchange with the target tissue is transported from the human body, and the remaining cold in the cold-carrying medium and the carrier flowing out of the medium storage tank are used. The cooling medium is exchanged in the second cooling capacity exchange device, so that the cooling medium that has not yet passed into the first cooling capacity exchange device is cooled and then transported to the first cooling capacity exchange device.

As a preferred technical solution, a part of the cold energy is stored in the pre cooling stage, and the cold energy is transferred to the cold carrier medium recovered from the duct, and flows out of the medium storage tank in the second cold energy exchange device. The cold carrier medium for cold capacity exchange.

As a preferred technical solution, in the rewarming step, the cooling medium is circuited through the heating device to raise the temperature of the cooling medium, and then the heated cooling medium is passed into the target tissue of the human body to make the cooling medium The medium exchanges heat with the target tissue, which heats up the target tissue after cooling.

As a preferred technical solution, in the rewarming step, a cold carrier that has not been refrigerated is input into the human body, so that the target tissue and the cold carrier that have not been refrigerated will heat up after heat exchange.

The technical scheme of the present intention has the following advantages:

1. The cryoablation device provided by the present invention includes a cryo-balloon, a catheter, a medium storage tank a medium supply pipeline, a medium recovery pipeline, and a refrigeration component; the cryo-balloon is filled with a cold medium, which is suitable for human tissue Contact and perform cryoablation; the catheter is connected with the cryo-balloon and has a medium input end and a medium output end, which is suitable for inputting and outputting the cold-carrying medium into the freezing balloon; the cold-carrying medium is stored in the medium storage tank, so The refrigerating medium is low-pressure liquid or gas or gas-liquid mixture; one end of the medium supply pipeline is connected with the medium storage tank, and the other end is connected with the medium input end of the pipe; one end of the medium recovery pipe is connected with the medium storage tank, and the other end is connected with the pipe The medium output end of the medium is connected; the refrigeration component is connected in series with the medium supply pipeline, which is suitable for providing cold energy to the cold medium in the medium supply pipeline. In the process of cryoablation, the cooling medium flows according to the following path: after flowing out of the medium storage tank along the medium supply pipeline, passing through the refrigeration component, the temperature of the cooling medium drops, and then enters the medium input end of the catheter. Then it flows into the freezing balloon to contact the target tissue via the balloon, and then flows out from the media output end of the catheter to the media recovery pipeline, and finally returns to the media storage tank to complete a cycle. In the above process, since the original high-pressure gas is replaced with a cold-carrying medium, the cold-carrying medium body is directly cooled and cooled, so that it can meet the low temperature requirements of freezing and ablation. Compared with high-pressure gas, the refrigerating medium is less prone to explosion, so it can effectively reduce the risk factor of the cryoablation device during use. At the same time, since the refrigerating medium can be recycled, there is no need to supplement high-pressure gas during use, which improves It is convenient to use. In addition, the low-pressure cold-carrying medium has a single cooling method, which only relies on refrigeration components for cooling. Therefore, its cooling process is easier to control then the cooling method that reset with high-pressure gas throttling, thus effectively reducing the difficulty of its operation. The chief surgeon does not need a professional technician Accompany you to perform the entire procedure.

2. In the cryoablation device provided by the present invention, the refrigeration assembly further includes a second cold quantity exchange device having a hot fluid channel installed on the medium supply pipeline and a cold fluid installed on the medium recovery pipeline Channel, the cold fluid channel and the hot fluid channel produce cold energy exchange, to pre-cool the cold medium flowing through the hot fluid channel; the hot fluid channel is connected between the medium storage tank and the The first cold capacity exchange device. During cryoablation, the medium supply pipeline, medium recovery pipeline, cryoablation system catheter and cryo-balloon are used to circulate the cold-carrying medium. During the flow, the cold generated by the cold generating device passes through the first The cold quantity exchange device is delivered to the medium supply and then delivered to the human body through the catheter to cryoablate the target tissue. The cold carrier medium that exchanges the supercooled quantity with the target tissue then flows from the catheter to the medium recovery At this time, the refrigerating medium still has part of the remaining cold capacity, and then when the refrigerating medium flows through the medium recovery pipeline, due to the existence of the second cold capacity exchange device, the remaining cold capacity in the refrigerating medium will pass through the second cold capacity The exchange device is conducted to the cooling medium in the medium supply pipeline, so that the cooling medium there is pre-cooled. In the above process, since the second cold quantity exchange device is upstream of the first cold quantity exchange device on the medium supply pipeline the temperature of the refrigerant-carrying medium in the medium supply pipeline is higher than the temperature in the medium recovery pipeline. Therefore, it can be ensured that the cold energy is conducted from the medium recovery pipeline to the medium supply pipeline. Through the above process, the remaining cold capacity of the cooling medium in the medium recovery pipeline can pre-cool the cooling medium, reducing the initial temperature of the cooling medium when it enters the first cooling capacity exchange device, and then at the same cooling capacity exchange capacity Under the circumstance, this pre-cooled cold carrier medium can reach a lower temperature, which makes the cryoablation method that directly refrigerates the low-pressure medium more likely to reach the temperature required for cryoablation, while also increasing the temperature Utilization efficiency of cold capacity.

3. The cryoablation device provided by the present invention further includes a bypass pipe which communicates with the medium supply pipe and the medium recovery pipe and makes the medium supply pipe and the medium recovery pipe form a series of media storage The tank and the pre-cooling circuit of the first refrigeration exchange device; and the bypass pipe and the medium supply pipeline communicate with each other through a first three-way valve. By using the bypass pipe the refrigerant can be pre-cooled before entering the human body for freezing and ablation. In the pre-cooling stage, the refrigerant will flow through the medium supply pipeline and the first cooling capacity after it comes out of the medium storage tank. Exchange device, bypass pipe and medium recovery pipeline, and finally return to the medium storage tank. The temperature of the cold-carrying medium after pre-cooling is reduced, and it has a lower initial temperature when entering the cryo-ablation stage, and after cooling by the first cold quantity exchange device, it is easier to reach the low temperature required for cryo-ablation. Therefore, this action can further increase the possibility that the cooling medium of the cryoablation device reaches the temperature required for cryoablation.

4. The freezing and ablation device provided by the present invention further includes a cold storage device, which is installed on the medium recovery pipeline and communicates with the first cold quantity exchange device by a bypass pipe which is suitable for storing the cold outflow from the first cold quantity exchange device. the amount. In the pre-cooling stage, the cold storage device can store part of the cold energy brought by the cold-carrying medium. After the freezing and ablation stage starts, the cold storage device can pre-cool the cold-carrying medium left from the human body to make the second The temperature difference between the cold fluid passage and the hot fluid passage at the cold quantity exchange device increases, and the cold quantity exchange rate at the second cold quantity exchange device is increased, thereby further reducing the temperature of the cold carrier medium in the medium supply pipeline. The pre-coded cooling medium can reach a lower temperature after being finally cooled by the first cooling capacity exchange device. Therefore, this action can further ensure that the cryoablation device can reach the low temperature required for cryoablation, and can further improve the cold energy utilization efficiency and reduce cold energy waste.

5. The freezing and ablation device provided by the present invention further includes a heat insulation device having a heat insulation cavity suitable for reducing or isolating heat conduction with the outside, and the first cold quantity exchange device, the second cold heat The cold output ends of the quantity exchange device, the cold storage device and the cold generation device are located in the heat insulation cavity. The use of the heat insulation device can avoid the low of cold energy during the cold energy exchange process, and at the same time, the heat preservation effect of the cold storage device is better, and the cold storage device can avoid the cold energy loss during the cold storage process.

6. The freezing and ablation device provided by the present invention the heat insulation device is a box, and a vacuuming device connected to the heat insulation cavity it installed on the heat insulation device. The heat-insulating cavity close to the vacuum state can further reduce the rate of loss of cold energy, so that the cryoablation device

7. The cryoablation device provided by the present invention further includes a rewarming circuit for holing the cold carrier medium in the storage tank and then delivering it to the medium input end of the catheter in the cryoablation device. In cryoablation, the frozen target tissue needs to be rewarmed. The ideal rewarming process can improve the surgical effect of cryoablation and reduce the probability of postoperative complications. The rewarming circuit provided in the present invention can heat the cold-carrying medium and deliver it to the target tissue through a catheter. This separately arranged rewarming circuit can not only meet the needs of cryoablation for rewarming, but also is very beneficial More precise control of the temperature, process and time of rewarming can improve the cure rate of surgery and reduce postoperative complications.

8. In the cryoablation device provided by the present invention, the rewarming circuit includes a rewarming tube, and a heating device is connected in series with the rewarming tube; the medium inlet end of the rewarming tube uses a second three-way valve to eater the station The hot fluid passage is connected upstream of the inlet port. After connecting, the reheating pipe to the upstream of the second cold exchange device on the medium supply pipeline, the reheating pipe is connected in parallel with the first cold exchange device and the second cold exchange device. At is time, the refrigerant used for heating The pipeline and the pipeline used for cooling are independent. Therefore, it is possible to avoid the residual cooling capacity of the first and second cooling capacity exchanging devices from interfering with the heating process of the carrier refrigerant dining the reheating stage, reducing interference factors in the rehearing process, and making the control of the reheating process more convenient control.

9. In the cryoablation device provided by the present invention, the rewarming circuit further includes a rewarming return has which is used to coot the medium output end of the catheter in the cryoablation device with the medium storage tank. The separate reheating return line can make the reheating process forma separate reheating circuit composed of a medium storage tack, a reheating tube, a cryoablation device, and a reheating return line, which can further reduce the interference factors in the reheating process, Make the process control of rewarming more precise.

10. The freezing and ablation device provided by the present invention further includes a heat insulation device, the heat insulation device having a hot insulation cavity suitable for reducing or isolating hot conduction with the outside, the first cold quantity exchange device, the second cold heat The cold output ends of the quantity exchange device, the cold storage device and the cold generation device are located in the heat insulation cavity; the reheating return pipe is outside the heat insulation device. After the reheating return pipe is arranged outside the heat insulation device, it can prevent the reheating return pipe from taking away the cold energy in the cold storage device or the second cold energy exchange device when transporting the reheated cold carrier medium, thereby increasing the cold energy Utilization rate.

11. The technical solution provided by the present invention also includes a cryo-ablation method, which includes the following steps: pre-cooling, posing a low-pressure cold-carrying medium cold cycle into the cold generation device for cooling; ablate, completing the pre-cooling The cold-carrying medium is circulated into the cold generation device, and then passed into the target tissue of the human body, so that the cold-carrying medium and the target tissue can exchange cold, so that the target tissue is cooled and the target tissue is cryoablated; Recover, transport the cold-carrying medium after the cold exchange with the target tissue from the human body, and transport it to the storage tank; rewarming, re-warming, stop passing the cooled cold-carrying medium into the human body to make the target To warm up the organization. In the above steps, due to the pre-cooling step, the cooling medium has a lower temperature after being pre-cooled, in that the initial temperature dining the cooling process in the ablation stage will be lower, so the temperature that can be finally reached will be lower. Compared with the prior art method of directly cooling the cold-carrying medium and then passing it into the human body, the cold-carrying medium in is method can more easily reach the low temperature required for cryoablation.

12. In the cryoablation method provided by the present invention, in the recovery step, the cold-carrying medium after the cold exchange with the target tissue is transported out of the human body, and the remaining cold in the cold-carrying medium and the non-passing cold are used. The cooling medium in the cooling capacity generating device exchanges the cooling capacity, so that the cooling medium that gas not been posed into the cooling capacity generating device is cooled and then transported to the cooling capacity generating device. In the above steps, the remaining cold energy of the refrigerant-carrying medium in the medium recovery pipeline can be reused, the utilization efficiency of the cold energy can be improved, and the power burden of the cold energy generating device can be reduced.

13. In the freezing and ablation method provided by the present invention, a part of the cold energy is stored in the pre-cooling stage, and the cold energy is transported to the cold-carrying medium sent from the human body to cool the cold-carrying medium sent from the human body. Through the above steps, the pre-stored cooling capacity can be transferred to the medium recovery pipeline to reduce the temperature of the cooling medium, so that the residual cooling capacity of the transported cooling medium can be used in the recovery step to cool down the unimported cooling medium The temperature difference in the process is larger, and the cooling capacity exchange speed between the two is increased, so that the temperature of the cooling medium that is not input into the human body is lower, and it is easier to reach the temperature required for freezing and ablation under the cooling of the cooling capacity device.

In summary, the cryoablation device and cryoablation method provided by the present invention have low risk factors, simple operation, convenient use, and at the same time have the advantages of high cold utilization efficiency, actuate temperature control and the like.

### DESCRIPTION OF THE DRAWINGS

In order to explain the specific embodiments of the present invention or the technical solutions in the prior art more clearly, the following will briefly introduce the drawings that need to be used in the specific embodiments or the description of the prior art. Obviously, the appendix in the following description The drawings are some embodiments of the present invention. For those of ordinary skill in the art, other drawings can be obtained based on these drawings without creative work.

FIG. 1 is a schematic diagram of the mechanism of the cryoablation device provided in Embodiment 1 of the present invention;

FIG. 2 is a schematic diagram of the flow of the cold carrier medium in the pre-cooling stage of the cryoablation device shown in FIG. 1;

FIG. 3 is a schematic diagram of the flow of the cold medium in the cryoablation device shown in FIG. 1 in the cryoablation stage;

FIG. 4 is a schematic diagram of the flow of the cold medium in the cryoablation device shown in FIG. 1 during the rewarming stage;

FIG. 5 is a flowchart of the cryoablation method provided in Embodiment 3 of the present invention;

### DESCRIPTION OF REFERENCE SIGNS

1—medium storage tank, 2—medium supply pipeline, 3—medium recovery pipeline, 4—cold generating device, 5—first cold-energy exchange device, 6—second cold-energy exchange device, 7—pipe, 8—Freezing balloon, 9—bypass pipe, 10—first three-way valve, 11—cold storage device, 12—heat insulation device, 13—hot insulation chamber, 14—vacuum device, 15—reheating tube, 16—Heating device, 17—second three-way valve, 18—reheating return pipe, 19—third three-way valve, 20—pumping device, 21—flow meter, 22—thermometer, 23—check valve, 24—hot dissipation Device.

### DETAILED WAYS

The technical solutions of the present invention will be clearly and completely described below in conjunction with the accompanying drawings. Obviously, the described embodiments are part of the embodiments of the present invention, rather than all of them. Based on the embodiments of the present invention, all other embodiments obtained by those of ordinary skill in the art without creative work shall fall within the protection scope of the present invention.

In the description of the present invention, it should be noted that the terms “center”, “upper”, “lower”, “left”, “right”, “vertical”, “horizontal”, “inner”, “outer”, etc. The indicated orientation or positional relationship is based on the orientation or positional relationship shown in the drawings, which is only for the convenience of describing the present purposes, and cannot be understood as indicating or implying relative importance.

In the description of the present invention, it should be noted that the term “installed”, “connected” and “connected” should be understood in a broad sense, unless otherwise clearly specified and limited. For example, they can be fixed or detachable. Connected or integrally connected; it can be a mechanical connection or an electrical connection; it can be directly connected or indirectly connected through an intermediate medium, and it can be the internal communication between two components. For those of ordinary skill in the art, the specific meaning of the above-mentioned terms in the present invention can be understood in specific situations.

In addition, the technical features involved in the different embodiments of the present invention described below can be combined with each other as long as they the not conflict with each other.

### Example 1

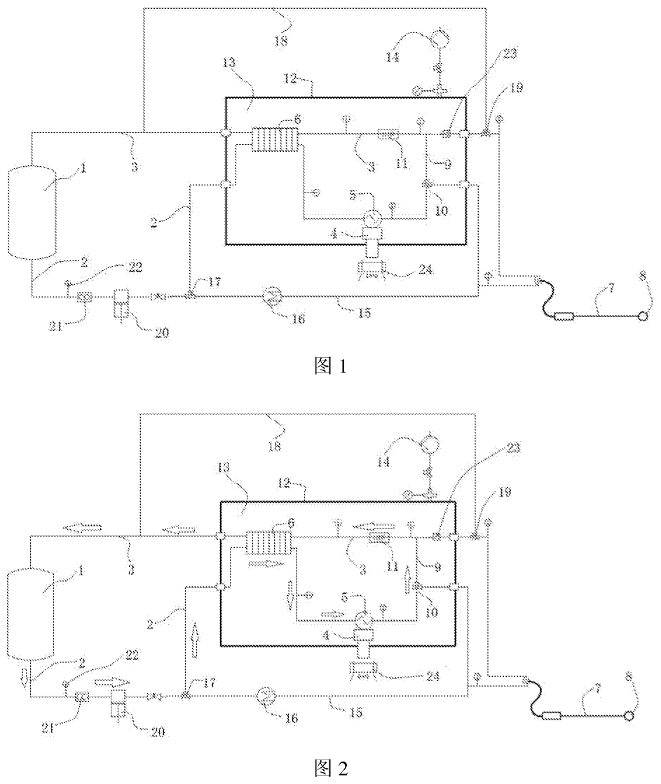

As shown in FIGS. 1 to 4, it is the first embodiment of the present invention. This embodiment provides a cryoablation device, which includes a cryo-balloon 8, a catheter 7, a medium storage tank 1, a medium supply line 2, and a medium recovery The pipeline 3 and the refrigeration components; the refrigerating balloon 8 circulates with a cold medium, which is suitable for contacting human tissue and performing cryoablation; the catheter 7 is connected to the refrigerating balloon 8 and has a medium input end and a medium output end, It is suitable for inputting and outputting cold-carrying medium into the freezing balloon 8; the cold-carrying medium is stored in the medium stooge tank 1, and the cold-carrying medium is a low-pressure medium; one end of the medium supply pipeline 2 is connected with the medium storage tank 1, and the other end The inlet end of the pipe 7 is connected; one end of the medium recovery pipe 3 is connected with the medium storage tank 1, and the other end is connected with the outlet end of the pipe 7; the refrigeration component is connected in series with the medium supply pipe 2 and is suitable for supplying the medium inside the pipe 2 The cooling medium is used for cooling and heat exchange.

In the process of cryoablation, the cooling medium flows according to the following path: after flowing out of the medium storage tank 1 along the medium supply pipeline 2, passing through the refrigeration components, the temperature of the cooling medium drops, and then enters the medium in the conduit 7 The input end then flows into the freezing balloon 8 through the balloon to contact the target tissue, and then flows out from the media output end of the catheter 7 to the media recovery pipeline 3, and finally returns to the media storage tank 1 to complete a cycle. In the above process, since the original high-pressure gas is replaced with the cold-carrying medium, the cold-carrying medium is directly refrigerated. Compared with the high-pressure gas, it is less prone to explosion, which can effectively reduce the risk of the cryoablation device during use At the same time, because the low-pressure cooling medium can be recycled, there is no need to supplement high-pressure gas during use, which improves its convenience in use. In addition, the low-pressure cold-carrying medium has a single cooling method, which only relies on refrigeration components for cooling. Therefore, the cooling process is easier to control than the cooling method that relies on high-pressure gas throttling, thus effectively reducing the difficulty of its operation. The chief surgeon does not need a professional technician Accompany you to perform the entire procedure.

As a specific implementation of the refrigeration assembly, the refrigeration assembly includes a refrigeration generating device 4, a first refrigeration exchange device 5, and a second refrigeration exchange device 6. The cooling capacity generating device 4 is used to provide cooling rapacity; the first cooling capacity exchanging device 5 is installed on the medium supply pipeline 2, and the cooling capacity generating device 4 provides cooling capacity to the first cooling capacity exchanging device 5. The first refrigeration exchange device 5 is used to perform refrigeration and heat exchange on the cooling medium passing through the first refrigeration exchange device 5; the second refrigeration exchange device 6 has heat installed on the medium supply pipe 2 The fluid channel and the cold fluid channel installed on the medium recovery pipeline 3, the cold fluid channel and the hot fluid channel generate cold energy exchange, and the cold medium flowing through the hot fluid channel Cold; the hot fluid channel is connected between the medium storage tank 1 and the first cold exchange device 5.

When the above-mentioned refrigeration component participates in cryoablation, the medium supply line 2, the medium recovery line 3, the catheter 7 of the cryoablation system, and the cryo-balloon 8 are used to make the cold-carrying medium circulate. The cold energy generated at 4 places is delivered to the medium supply pipeline 2 through the first cold energy exchange device 5, and then delivered to the human body through the catheter 7 for cryoablation of the target tissue, and the carrier for supercooling exchange with the target tissue. The cold medium then flows from the conduit 7 to the medium recovery pipeline 3. At this time, the cold carrier medium still has some cold capacity. Then, when the cold carrier flows through the medium recovery line 3, due to the second cold exchange device 6 With the existence of, the remaining cold in the cold-carrying medium will be transferred to the cold-carrying medium in the medium supply pipeline 2 through the second cold-exchanging device 6, so that the cold-carrying medium there is pre-cooled.

In the above process, since the second cooling capacity exchange device 6 is upstream of the first cooling capacity exchange device 5 on the medium supply pipe 2, the temperature of the cooling medium in the medium supply pipe is higher than that of the medium recovery. The temperature in the pipeline 3 can thus ensure that the cold energy is conducted from the medium recovery pipeline 3 to the medium supply pipeline 2. Through the above process, the remaining cold capacity of the cooling medium in the medium recovery pipeline 3 can pre-cool the cooling medium, reduce the initial temperature of the cooling medium when it enters the first cooling capacity exchange device 5, and then at the same cooling capacity In the case of exchange volume, this pre-cooled cold carrier medium can reach a lower temperature, so that this cryoablation method that directly cools the low-pressure medium is more likely to reach the temperature required for cryoablation, and at the same time Improve the efficiency of cold utilization.

Specifically, the cold generation device 4 in this embodiment is specifically a miniature ultra-low temperature refrigerator capable of providing a cold source below −120° C., which can be in the form of pulse tube, Stirling, mixed working fluid throttling, thermoacoustic, etc. It can be one or more units. When multiple units work together, the joint mode can be series or parallel. The cooling medium in this embodiment is a medium with a low freezing point, such as absolute ethanol.

In order to further reduce the minimum temperature that the cooling medium can reach, it also includes a bypass pipe 9, which communicates with the medium supply pipe 2 and the medium recovery pipe 3, and allows the medium supply pipe 2 and The medium recovery pipeline 3 forms a pre-cooling circuit connecting the medium storage tank 1 and the first cooling capacity exchange device 5 in series; and the bypass pipe 9 and the medium supply pipeline 2 are in communication through the first three-way valve 10.

By using the by-pass pipe 9, the refrigerant can be pro-cooled before entering the human body for freezing and ablation. In the pre-cooling stage, the refrigerant will flow through the medium supply pipeline 2 and the second after coming out of the medium storage tank 1. A cold capacity exchange device 5, a bypass pipe 9 and a medium recovery pipeline 3, and finally return to the medium storage tank 1. The temperature of the cold carrier medium after pre-cooling is reduced, and has a lower initial temperature when entering the cryoablation stage, and after cooling by the first cold quantity exchange device 5, it is easier to reach the low temperature required for cryo-ablation. Therefore, this action can further increase the possibility that the cryoablation device is carrying the cold medium to reach the temperature required for cryoablation.

As an improved embodiment of the cryo-ablation device, it also includes a cold storage device 11, which is installed on the medium recovery pipeline 3, and communicates with the first cold energy exchange device 5 by a bypass pipe 9, suitable for storing the first cold energy exchange T, amount of cold flowing out of the device 5. In this embodiment, the cold storage device 11 is specifically a box filled with a cold storage medium with a higher specific heat opacity. The medium recovery pipeline 3 passes through the cold storage device 22, and the side wall of the pipeline and the cold storage medium in the cold storage device 11 are used to generate cold energy exchange.

In the pre-cooling stage, the cold storage device 11 can store part of the cold energy brought by the cold-carrying medium. After the freezing and ablation stage starts, the cold storage device 11 on pre-cool the cold-carrying medium left from the human body to make the first The temperature difference between the cold fluid passage and the hot fluid passage of the second cold quantity exchange device 6 is increased, which increases the cold quantity exchange rate at the second cold quantity exchange device 6, thereby further increasing the temperature of the cold medium in the medium supply pipeline 2 Lowering, these pre-cooled cooling medium on reach a lower temperature after the first cooling capacity exchange device 5 undergoes float cooling. Therefore, this action can further ensure that the cryoablation device can reach the low temperature required for cryoablation, and can further improve the cold energy utilization efficiency and reduce cold energy waste.

In order to reduce the loss of cold energy, it also includes a heat insulation device 12, the heat insulation device 12 has a heat insulation cavity 13 suitable for redoing or exchange process, and at the same time, the heat preservation effect of the cold storage device 11 is better, and the cold storage device 11 can avoid cold energy loss during the cold storage process.

Specifically, the heat insulation device 12 is a box, and a vacuum device 14 communicating with the heat insulation cavity 13 is installed on the heat insulation device 12. The heat-insulating cavity 13 close to the vacuum state can further reduce the loss rate of cold energy, so that the cryoablation device further improves the utilization efficiency of cold energy. The vacuum pump 14 is specifically a small vacuum pump.

As another alternative implementation of the heat insulation device, the heat insulation device 12 is a box, and the heat insulation cavity 13 is filled with a heat insulation material, where the heat insulation material may be a polyurethane foam material or aerogel material.

In order to meet the needs of the target tissue during cryoablation for rewarming after freezing, this embodiment also includes a rewarming circuit for heating the cold carrier medium in the storage tank and then transporting it to freezing The media input end of the catheter 7 in the ablation device. In cryoablation, the frozen target tissue needs to be rewarmed. The ideal rewarming process can improve the surgical effect of cryoablation and reduce the probability of postoperative complications. The rewarming circuit provided in the present invention can heat the cold-carrying medium and transport it to the target tissue through the catheter 7. This separately arranged rewarming circuit can not only meet the needs of cryoablation for rewarming, but also is very useful It is conducive to more precise control of the temperature, process and time of rewarming, thereby increasing the surgical cure rate and reducing postoperative complications.

Specifically, the reheating circuit includes a reheating pipe 15 which is connected in series with a heating device 16; the medium inlet end of the reheating pipe 15 uses a second three-way valve 17 and enters the hot fluid The inlet of the channel is connected upstream. After the reheating pipe 15 is connected to the upstream of the second refrigeration exchange device 6 on the medium supply pipeline 2, the reheating pipe 15 is connected in parallel with the first refrigeration exchange device 5 and the second refrigeration exchange device 6. The pipeline used for heating the refrigerant and the pipeline used for cooling are independent of each other. Therefore, it is possible to avoid the residual cooling capacity of the first refrigeration exchange device 5 and the second refrigeration exchange device 6 from interfering with the heating process of the easier refrigerant during the rewarming stage, reduce the interference factors of the rewarming process, and control the rewarming process It is easier to control.

As an alternative implementation of the above reheating circuit, the reheating circuit includes: a reheating pipe 15; the medium inlet end of the reheating pipe 15 is installed on the medium supply pipeline 2 by using a second three-way valve 17 The side that does not enter the first cold quantity exchange device 5 is connected. In this alternative embodiment, there is no series heating device on the rewarming circuit, but only the uncooled cold easier medium is introduced into the catheter to participate in the rewarming process, and the body's own heat is used for rewarming. This makes the temperature rise of the targeted tissues more gentle and reduces the damage to healthy tissues caused by cryoablation.

As a further improvement of the reheating circuit, the reheating circuit also includes a reheating return line, which is used to connect the medium output end of the catheter 7 in the cryoablation device with the recovery port of the medium storage tank 1 Connected. The separate reheating return line can make the reheating process form a separate reheating circuit composed of the medium storage tank 1, the reheating tube 15, the cryoablation device, and the reheating return line, thereby further reducing the temperature during the reheating process. Interference factors make the process of rewarming more precise.

Specifically, the reheating return pipe includes a reheating return pipe 18, both ends of which are connected with the medium recovery pipe 3 and connected in parallel with the second cold quantity exchange device 6; the medium inlet end of the reheating return pipe 18 is used The third three-way valve 19 is connected to the medium supply pipeline 2. Further, the reheating return pipe 18 is located outside the heat insulation device 12. After the reheating return pipe 18 is arranged outside the heat insulation device 12, it can prevent the reheating return pipe 18 from taking away the cold storage device 11 or the second cold energy exchange device 6 when the reheating refrigeration medium is transported. Increase the utilization rate of cooling capacity.

In order to ensure the smooth circulation of the cooling medium, a pumping device 20 is connected in series to the medium supply pipeline 2 or the medium recovery pipeline 3, and the pumping device 20 is adapted to provide power for the flow of the cooling medium.

The cryoablation device provided in this embodiment also includes an operating device. The operating device includes a handle and an actuator for operating the catheter 7 to reach the target tissue for ablation. When performing cryoablation, the catheter 7 is a pipe that transports low-temperature cold-carrying medium, and is made of a material with a certain degree of toughness, a small thermal conductivity, and physiological compatibility. It has multiple flow channels inside, which are respectively The inlet and outlet flow channels, functional channels and isolation chambers of cold media. The cooling medium inlet and outlet flow channels are distributed on both sides to insulate the inlet and outlet fluids and avoid thermal short circuits. The functional channel is located in the center of the catheter 7 and is used for routing of functional components such as sensors and guidewires. The isolation cavities are distributed at both ends of the cold medium flow channel to further reduce the heat exchange of the fluid in and out. The tube 7 is wrapped with thermal insulation material to reduce the heat transfer between the cold-carrying medium in the duct 7 and external human tissues, on the one hand, to reduce the heat leakage of the cold-carrying medium, and on the other hand, to avoid freezing of the tissue caused by the low temperature of the outer wall of the duct 7 The cryo-balloon 8 is used for cryoablation of the target tissue, and has a medium inlet and a medium outlet inside. The medium inlets connected with the inlet of the catheter 7 and the outlet of the medium is connected with the outlet of the catheter 7. After the balloon contacts the tissue, the cold-carrying medium exchanges heat with the tissue through the balloon wall.

### Example 2

As shown in FIG. 5, it is the second embodiment of the present invention. This embodiment provides a cryoablation method. In this method, a cold-carrying medium using a low-pressure medium is directly passed into the human body after refrigeration to perform targeted tissues on the human body. Cryoablation includes the following steps:

For pre-cooling, a low-pressure cooling medium is passed into the cooling capacity generating device 4 for cooling.

In ablation, the pre-cooled cold-carrying medium is circulated into the cold generation device 4, and then passed into the target tissue of the human body, so that the cold-carrying medium and the target tissue can exchange cold, so that the target tissue is cooled Then the target tissue is cryoablated.

Recycling, transport the cold-carrying medium after cold exchange with the target taste from the human body and transport it to the storage tank.

After rewarming, stop passing the cooled cold carrier medium into the human body to raise the temperature of the target tissue.

In the above steps, due to the pre-cooling slip, the cooling medium has a lower temperature after being pre-cooled, so that the initial temperature during the cooling process in the ablation stage will be lower, so the temperature that can be finally reached will be lower. Compared with the prior art method of directly cooling the cold-carrying medium and then passing it into the human body, the cold-carrying medium in this method can more easily reach the low temperature required for cryoablation.

In the recovery step, the cold-carrying medium after the cold exchange with the target tissue is transported from the human body, and the remaining cold in the cold-carrying medium and the cold-carrying medium that has not been passed into the cold generating device 4 are used for cooling. The quantity exchange, the cooling medium that has not yet passed into the cooling capacity generating device 4 is cooled and then transported to the cooling capacity generating device 4. In the recovery step, the remaining cold energy of the cooling medium in the medium recovery pipeline 3 can be reused, the utilization efficiency of the cold energy can be improved, and the power burden of the cold energy generating device 4 can be reduced.

In the pre-cooling step, a part of the cold energy is stored, and the cold energy is transported to the cold-carrying medium sent from the human body to cool the cold-carrying medium sent from the human body. Through the above steps, these pre-stored refrigeration can be transferred to the medium recovery pipeline 3 to reduce the temperature of the refrigerating medium, so that the residual refrigeration of the conveyed refrigerating medium can be used in the recovery step to perform The temperature difference in the

As a specific embodiment of the rewarming step, in the rewarming step, the cooling medium is circulated through the heating device (16), the cooling medium is heated, and then the heated cooling medium is passed into the target of the human body At the tissue, the cold-carrying medium and the target tissue generate heat exchange, so that the temperature of the target tissue after cooling is raised.

As an alternative implementation of the above-mentioned rewarming step, in the rewarming step, a cold-carrying medium that has not been refrigerated can also be input into the human body, so that the target tissue can exchange heat with the cold-carrying medium that has not beet refrigerated and then increase in temperature.

In combination with the cryoablation device in Embodiment 1, the specific process of the cryoablation method in this embodiment is:

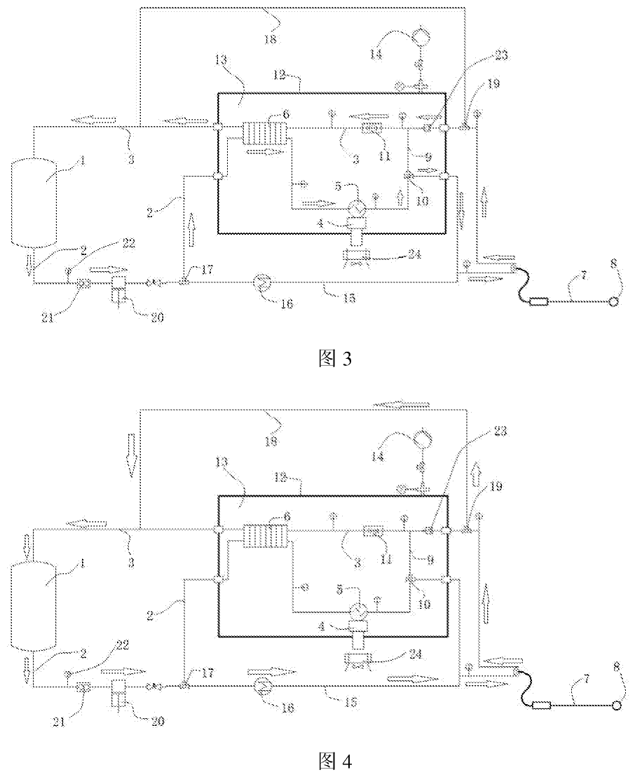

In the pre-cooling stage, see FIG. 2. The cooling capacity generating device 4 and the pumping device 20 are opened, and the three three-way valves are adjusted to make the cooling medium flow according to the following process: After the normal temperature cooling medium flows out of the medium storage tank 1, After passing through the flow meter, the pumping device 20, and the flow regulating valve, it enters the heat insulation device 12. After passing through the second refrigeration exchange device 6 to exchange heat with the refluxing refrigerating medium for pre-cooling, it enters the first refrigeration exchange device 5 for cooling, After passing through the bypass pipe 9, it enters the cold storage device 11, stores a part of the cold energy in the cold storage device 11, and then flows back to the second cold energy exchange device 6 to pre-cool the cold carrier medium left from the medium storage tank 1, and finally It flows out of the heat insulation device 12 and finally returns to the medium storage tank 1.

After about 20 minutes of pre-cooling cycle, the cooling medium drops to minus 80-100° C.

In the ablation stage, refer to FIG. 3. After the pre-cooling is completed, adjust the three three-way valves so that the cooling medium flows according to the following process: after passing through the flow meter, the pumping device 20, and the flow regulating valve, it enters the insulation device 12, and passes through the first After the second cooling capacity exchange device 6 exchanges heat with the refluxed cooling medium, it enters the first cooling capacity exchange device 5 for cooling. After the cooling medium is cooled by the first cooling capacity exchange device 5, it flows out of the heat insulation device 12 and enters the duct 7. Then enter the freezing balloon 8 for ablation, then flow out of the catheter 7, return to the heat insulation device 12, and flow through the cold storage device 11. At this time, the temperature of the cold carrier medium is higher than the temperature of the heat storage medium in the cold storage device 11. The heat storage medium releases heat and the temperature drops, and then enters the second cooling capacity exchange device 6 to exchange cooling capacity for the cooling medium flowing out of the medium storage tank 1, then exits the heat insulation device 12 and returns to the medium storage tank 1.

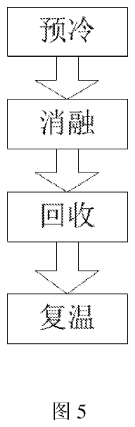

In the rewarming stage, refer to FIG. 4. After the freezing and ablation is completed, adjust the three three-way valves to make the cooling medium flow according to the following process: the cooling medium first comes out of the medium storage tank 1 and flows through the flow meter and the pumping device 20 After the flow control valve, it enters the heating device 16 and is heated to 37° C. by the heating device 16, and then enters the catheter 7 through the rewarming tube 15, heating and rewarming the ablated tissue, and flows out of the catheter 7 and passes through the third three-way The valve 10 flows through the reheating return pipe 18 and then returns to the medium storage tank 1.

Obviously, the above-mentioned embodiments are merely examples for clear description, and are not intended to limit the implementation. For those of ordinary skill in the art, other changes or changes in different forms can be made on the basis of the above description. It is unnecessary and impossible to list all the implementation methods here. The obvious changes or modifications derived from this are still within the protection scope created by the present invention.

1-21. (canceled)

22. A cryoablation apparatus comprising:
a cryoballoon, provided with a circulating cold carrier medium therein, and adapted for contacting a human tissue and performing cryoablation on the human tissue;
a catheter, connected to the cryoballoon, having an inlet end and an outlet end, and adapted for transporting the cold carrier medium into and out of the cryoballoon;
a storage tank, provided with the cold carrier medium stored therein;
a delivery pipeline, having one end communicated with the storage tank and another end communicated with the inlet end of the catheter;
a recovery pipeline, having one end communicated with the storage tank and another end communicated with the outlet end of the catheter; and
a refrigeration assembly, connected in series with the delivery pipeline, and adapted for cooling the cold carrier medium in the delivery pipeline.

23. The cryoablation apparatus of claim 22 wherein the refrigeration assembly comprises:
a first cold energy exchanger, mounted on the delivery pipeline and used for cooling the cold carrier medium flowing through the first cold energy exchanger by heat exchange; and
a cold energy generator, for generating cold energy and providing the cold energy to the first cold energy exchanger.

24. The cryoablation apparatus of claim 23 further comprising a bypass pipe communicated with the delivery pipeline and the recovery pipeline to allow the delivery pipeline and the recovery pipeline to form a precooling looping path connecting the storage tank and the first cold energy exchanger in series, wherein the bypass pipe is communicated with the delivery pipeline via a first three-way valve.

25. The cryoablation apparatus of claim 23 wherein the refrigeration assembly further comprises a second cold energy exchanger having a hot fluid channel mounted on the delivery pipeline and a cold fluid channel mounted on the recovery pipeline, wherein cold energy exchange occurs between the cold fluid channel and the hot fluid channel to precool the cold carrier medium flowing through the hot fluid channel, and wherein the hot fluid channel is connected between the storage tank and the first cold energy exchanger.

26. The cryoablation apparatus of claim 25 wherein the refrigeration assembly further comprises a cold storage device, mounted on the recovery pipeline, communicated with the first cold energy exchanger via the bypass pipe, and adapted for storing cold energy coming from the first cold energy exchanger.

27. The cryoablation apparatus of claim 26 wherein the refrigeration assembly further comprises a heat-insulation device, having a heat-insulation chamber adapted for reducing or eliminating heat conduction to the exterior thereof, wherein the first cold energy exchanger, the second cold energy exchanger, the cold storage device, and a cold energy output end of the cold energy generator are located inside the heat-insulation chamber.

28. The cryoablation apparatus of claim 27 wherein the heat-insulation device is a box mounted with a vacuumizing device communicated with the heat-insulation chamber.

29. The cryoablation apparatus of claim 27 wherein the heat-insulation device is a box with the heat-insulation chamber thereof filled with heat insulation substance.

30. The cryoablation apparatus of claim 23 further comprising a rewarming looping path for transporting the cold carrier medium in the storage tank to the inlet end of the catheter of the cryoablation apparatus.

31. The cryoablation apparatus of claim 30 wherein the rewarming looping path comprises a rewarming pipe, and wherein an inlet end of the rewarming pipe is connected by a second three-way valve to a side of the delivery pipeline that is located upstream of the first cold energy exchanger.

32. The cryoablation apparatus of claim 23 further comprising a rewarming looping path for transporting the cold carrier medium in the storage tank to the inlet end of the catheter of the cryoablation apparatus after heating the cold carrier medium.

33. The cryoablation apparatus of claim 32 wherein the rewarming looping path comprises a rewarming pipe with a heating device connected in series, and wherein an inlet end of the rewarming pipe is connected by a second three-way valve to an upstream side of an inlet to the hot fluid channel.

34. The cryoablation apparatus of claim 30 wherein the rewarming looping path also comprises a rewarming backflow pipeline for connecting the outlet end of the catheter of the cryoablation apparatus to the storage tank.

35. The cryoablation apparatus of claim 34 wherein the rewarming backflow pipeline comprises a rewarming backflow pipe having both ends thereof communicated with the recovery pipeline and being connected in parallel to the second cold energy exchanger, and wherein an inlet of the rewarming backflow pipe is connected by a third three-way valve to the delivery pipeline.

36. The cryoablation apparatus of claim 35 wherein the refrigeration assembly further comprises a heat-insulation device having a heat-insulation chamber adapted for reducing or eliminating heat conduction to the exterior thereof, wherein the first cold energy exchanger, the second cold energy exchanger, a cold storage device, and a cold energy output end of the cold energy generator are located inside the heat-insulation chamber, and wherein the rewarming backflow pipe is located outside the heat-insulation device.

37. A cryoablation method comprising:
a precooling step, in which a cold carrier medium is circulated through a cold energy generator for cooling;
an ablating step, in which the precooled cold carrier medium is re-circulated through the cold energy generator for re-cooling and then is transported to a target tissue of a human body, so that cold energy exchange occurs between the cold carrier medium and the target tissue to cool the target tissue for cryoablation of the target tissue;
a recovering step, in which the cold carrier medium after cold energy exchange with the target tissue is transported out of the human body and into a storage tank; and
a rewarming step, in which the transportation of the cooled cold carrier medium into the human body is stopped, and the target tissue is rewarmed.

38. The cryoablation method of claim 37 wherein, in the recovering step, the cold carrier medium after cold energy exchange with the target tissue is transported out of the human body, and residual cold energy in the cold carrier medium transported out of the human body is utilized to carry out cold energy exchange with the cold carrier medium flowing out of the storage tank at a second cold energy exchanger, so as to cause the cold carrier medium before entry into the first cold energy exchanger to be cooled and then transported into the first cold energy exchanger.

39. The cryoablation method of claim 37 wherein a part of cold energy is stored in the precooling stage, and then transferred to the cold carrier medium recovered from a catheter, so as to be exchanged to the cold carrier medium flowing out of the storage tank at a second cold energy exchanger.

40. The cryoablation method of claim 37 wherein, in the rewarming step, the cold carrier medium is circulated through a heating device for being warmed, and then the cold carrier medium after being warmed is transported to the target tissue of the human body, so as to warm the cooled target tissue by heat exchange between the cold carrier medium and the target tissue.

41. The cryoablation method of claim 37 wherein, in the rewarming step, uncooled cold carrier medium is transported to the human body, so as to warm the target tissue by heat exchange between the uncooled cold carrier medium and the target tissue.
